(19) United States
(12) Patent Application Publication
(10) Pub. No.: US 2008/0248563 A1
(43) Pub. Date: Oct. 9, 2008

(54) Specimen container for the micro manipulation and biopsy in in-vitro fertilization

(75) Inventors: Michael D. Cecchi, Madison, CT (US); Jacques Cohen, New York, NY (US); Tim Shimmel, Randolph, NJ (US)

Correspondence Address: William W. Jones, 6 Juniper Lane, Madison, CT, 06443, US

(73) Assignee: Genx International, Inc.

(21) Appl. No.: 11/732,187

(22) Filed: Apr. 4, 2007

(51) Int. Cl. C12M 1/28

(52) U.S. Cl. 435/309.1

(57) ABSTRACT

A container or dish is useful for the micro manipulation, micro injection, biopsy and fertilization in oocyte and embryo manipulation and culturing. The dish allows the user to more readily perform procedures used to fertilize oocytes by Intracytoplasmic sperm injection (ICSI) or other procedures, biopsy embryos and perform additional procedures for use in assisted reproductive techniques (ART), human reproduction and in-vitro fertilization (IVF). The invention allows ease in use, the reduction in the number of micro tools used in the procedure, as opposed to conventional dishes and procedures, and will allow the user to more readily locate the oocytes and embryos to be handled and worked on. The invention will add repetitiveness, consistency, improve efficacy and ease of procedure and may improve outcomes. The invention will also give the user a more ergonomically practical dish for these types of procedures and related protocols. Specimens other than oocytes and embryos can be treated in the container by means of micro manipulation.

### TECHNICAL FIELD

This invention relates to a container or culture dish that is to be used for the preparation and to allow the user to complete certain procedures on the specimens. The dish allows the user to perform multiple procedures, such as micro manipulations, micro injection and biopsies on the specimens such as oocytes, spermatozoa, embryos and stem cells with greater ease, in less time and allowing more productivity. The dish includes multiple depressions in its bottom surface which create a unique working configuration. The depressions provide ease in locating the specimens as well as the ability to perform such multiple procedures, also resulting in the use of fewer micro manipulation tools and instruments, than are now used to perform similar procedures. The dish can be used for a wide range of specimens of animal and human cells, tissues, stem cells, embryos, oocytes, immature oocytes, sperm precursor cells, embryonic cells, blastocysts, and spermatozoa with all such specimens receiving the same benefits.

### BACKGROUND ART

Current devices, containers or culture dishes used for procedures such as micro manipulation and are used for Intracytoplasmic sperm injection (ICSI) are not unique and are mostly generic, off-the-shelf, dishes used for these procedures. The micro manipulation of immature oocytes, mature oocytes, gametes, zygotes, embryos, cleavage stage embryos, blastocyst stage embryos, precursor cells, and such, referred to herein as “specimens”, are done in standard 30 mm, 50 mm or 60 mm Petri dishes, with no unique features, that simply have flat bottoms and lids for storage purposes.

One problem with using generic Petri dishes in micro manipulation of specimen procedures is that they do not have unique dish configurations which enhance their utility in such procedures. Another problem with the current dishes is that they require the user to use micro drops of a specimen medium solution on the surface of the dish. Some dishes may create surface tension problems with the specimen medium solution. This can result in the micro drops collapsing and the media solution that the sample is in becomes compromised by an overlain layer of oil. The reverse may happen too and the droplets may not adhere well to the bottom of the dish causing them to move in the dish. Yet another problem with the current dishes is that for a user to perform an ICSI or other micro manipulation procedure such as biopsy, assisted hatching etc., procedure they must use a holding pipette and ICSI pipette or other micro tool. The holding pipette is for holding the specimen in place in the dish and the ICSI pipette is for injection of something into the specimen in the dish, for example, for injecting sperm into an oocyte for IVF fertilization procedure. This requires at least two pipettes to perform the procedure which increases costs and results in extra work required to set up two tools for each procedure. Yet another problem incurred by use of current off the shelf dishes is that the user has difficulty in locating the specimen in the dish. Thus time must be spent locating the specimen and then capturing the specimen with the holding pipette or moving it under a microscope or correcting its position in the dish.

It would be highly desirable to provide a specimen treatment dish that will inherently locate the specimen being treated in a definite predetermined location in the dish and retain the specimen in that definite location.

### DISCLOSURE OF THE INVENTION

This invention relates to an improved container or dish and method for using the invention for the micro manipulation, micro injection and/or biopsy of spermatozoa, sperm precursor cells, oocytes, pre-implantation embryos, zygotes, blastocysts, embryonic cells and stem cells, collectively referred to hereinafter as “specimen” or “specimens”. The dish consists of a plurality of wells, each having a precisely configured side walls, base or floor which will hold the specimen in place and allow the user to work on the specimen. The dish will also allow the user to readily locate the specimens within the dish. The wells are configured to allow the user to readily move about the dish, locate samples and be able to perform the micro manipulation procedures more quickly and concisely. The sizes, shapes and heights all work together in harmony to allow the user to more effectively perform certain delicate procedures with more quickly and with more predictable and more accurately controlled results.

The following is a listing of several desirable objects of this invention.

It is an object of this invention to provide an improved container or dish for the micro manipulation or micro injection of specimens while improving speed, ease of procedure, efficacy and possibly clinical outcome.

It is another object of this invention is to provide a container or dish of the character described for the biopsy of embryos and obtaining stem cells more quickly and more accurately.

It is a further object of this invention to provide a dish of the character described which allows the user to reduce costs by reducing the number of instruments and time needed to perform the procedures.

It is a further object of this invention to provide an improved dish of the character described which allows the user to learn procedures faster by reducing the number of steps needed to perform micro manipulation.

It is a further object of this invention to provide an improved dish of the character described which allows the user to locate the cells faster and/or use an automated tool aimed at micro manipulating robotically as the object specimen will be locate at predetermined coordinates.

### BRIEF DESCRIPTION OF THE DRAWINGS

These and other objects and advantages of the invention will become more readily apparent from the following detailed description of several embodiments of the invention when taken in conjunction with the accompanying drawings in which:

FIG. 1 is a top plan view of a specimen dish formed in accordance with this invention; and

FIG. 2 is a fragmatic side sectional view of one of the well structures in the dish.

### DETAILED DESCRIPTION OF SPECIFIC EMBODIMENTS OF THE INVENTION

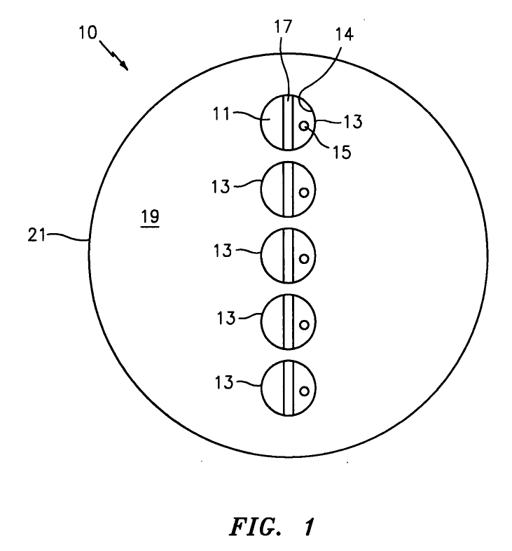

FIG. 1 is a top plan view of a specimen handling dish formed in accordance with this invention. The dish 10 includes an outer wall 21 and a top surface 19. Within the dish 10 are a plurality of wells 13. The wells 13 each have an outer wall 14 and a declining bottom wall 11 with a recessed basin 17 therein. Within each of the wells 14 there is a raised post 15 which can be used to release the specimen from a micro tool. The specimen will settle into and remain in the basin 17. The specimens in the wells 14 can will be immersed in a media solution with a oil overlay, for sustaining the specimens.

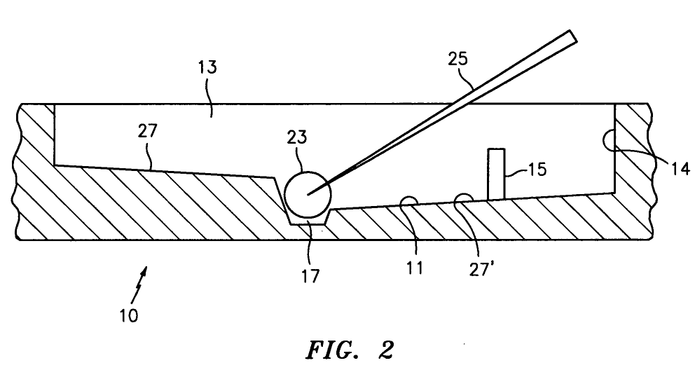

Referring now to FIG. 2, details of the interior of each of the wells 13 are shown. The bottom wall 11 of each well 13 is formed with two separated downwardly declining components 27 and 27′, one of which is taller than the other. The post 15 is used to release the specimen 23 from a micro tool 25, by drawing the specimen 23 against the post 15 to aid in its release from the micro tool 25. The released specimen 23 will then migrate into the recessed basin 17 where it will remain. This allows the user to utilize the taller component 27 to hold the specimen 23 in place while pushing a micro tool 25 against the specimen 23 in order to penetrate the specimen 23. The basin 17 is sized to allow the specimen 23 to settle at the basin 17 and to be held in place by the components 27 and 27′. It will be noted that the micro tool 25 is shown schematically, and can be any number of tools, such as a suction catheter which can insert the specimen 23 into the well 13, or a syringe which can inject material into the specimen 23, or can remove material from the specimen, by way of example of various micro tools that can be used in conjunction with the dish 10 of this invention.

It will be appreciated that the dish of this invention can be used in micro manipulation procedures involving minute specimens such as single cells, embryos, stem cells, and the like. The specimen in question can be deposited in a well in the dish and will be located in a definite position in the well where it will be relatively immobilized for treatment and/or analysis. The wells can be used for culturing the specimens during the procedures involving the micro manipulation of the specimens.

While the invention has been described with respect to preferred embodiments, those skilled in the art will readily appreciate that various changes and/or modifications can be made to the invention without departing from the spirit or scope of the invention as defined by the appended claims.

What is claimed is:

1. A mammalian cell specimen manipulation or biopsy apparatus comprising:
a) a container for holding the specimens, and for holding a culturing medium which sustains the specimens during the procedure, said container having a bottom wall; and
b) at least one specimen gathering structure in said container, said structure being positioned on said bottom wall of said container, and said structure being sized to capture single ones of the specimens.

2. The apparatus of claim 1 wherein said structure is disposed between opposed downwardly and inwardly angled portions of said bottom wall, said portions being operative to direct specimens in said container toward said structure.

3. The apparatus of claim 2 wherein said structure is a slot having a bottom surface and opposed side surfaces that intersect said portions of said container bottom wall.

4. The apparatus of claim 3 wherein one of said opposed side surfaces is taller than the other.

5. The apparatus of claim 3 further comprising a post structure on one of the angled portions of said bottom wall which post structure is operative to dislodge a specimen from a micro tool that is inserted into said container so that the dislodged specimen will settle into said specimen gathering structure in said container.

6. The apparatus of claim 1 wherein said container is a dish having a side wall, a bottom wall and a top wall, and which includes a plurality of wells in said top wall, each of which wells containing a separate specimen gathering structure whereby multiple specimens can be segregated and treated in said container, one in each of said wells.
